(19) United States
(12) Patent Application Publication
(10) Pub. No.: US 2024/0342003 A1
(43) Pub. Date: Oct. 17, 2024

(54) INTRAOCULAR PRESSURE FLUCTUATION CANCELLATION

(71) Applicant: JOHNSON & JOHNSON SURGICAL VISION, INC., Irvine, CA (US)

(72) Inventors: Assaf Govari, Haifa (IL); Vadim Gliner, Haifa (IL); Alon Boumendil, Givat Nili (IL); Andres Claudio Altmann, Haifa (IL)

(21) Appl. No.: 18/135,208

(22) Filed: Apr. 17, 2023

(51) Int. Cl. A61F 9/007

(52) U.S. Cl. CPC A61F 9/00745; A61B 2017/00973; A61B 2090/064; A61B 2217/005; A61B 2217/007

(57) ABSTRACT

A phacoemulsification system, including a probe having an irrigation channel and an aspiration channel, and a distal end including a needle and a sleeve insertable into an eye. There is an irrigation pump pumping irrigation fluid via the irrigation channel into the eye, and an aspiration pump pumping aspiration fluid via the aspiration channel from the eye. A first pressure sensor is coupled with the irrigation fluid and provides a first signal of intraocular pressure (IOP) in the eye; a second pressure sensor is coupled with the aspiration fluid and provides a second signal of the IOP. A system processor receives the first and second signals, and responsively to at least one of the signals, identifies at least one frequency and an associated at least one phase of the IOP, and pumps at least one of the pumps at the identified frequency and in antiphase to the identified phase.

### FIELD OF THE DISCLOSURE

This disclosure relates generally to ocular surgery, and specifically to changes in the intraocular pressure (IOP) that may occur during the surgery.

### BACKGROUND

A cataract is a cloudy area in the lens of the eye that leads to a decrease in vision. Phacoemulsification is a modern cataract surgery method in which the eye's internal lens is emulsified with an ultrasonic handpiece and aspirated from the eye. The aspirated fluids may be replaced by irrigation of the eye with a balanced salt solution to maintain the intraocular pressure (IOP) of the eye. Even so, during the surgery the IOP typically varies.

### BRIEF DESCRIPTION OF THE DRAWINGS

The present disclosure will be understood from the following detailed description, taken in conjunction with the drawings in which:

FIG. 1 is a pictorial view of a phacoemulsification apparatus, according to an example of the present disclosure;

FIG. 2 is a schematic block diagram illustrating an irrigation sub-system and an aspiration sub-system, according to an example of the present disclosure;

FIG. 3A is a flowchart of steps performed during a handpiece priming phase of a phacoemulsification procedure, and FIG. 3B is a schematic block diagram of elements used during the priming phase, according to an example of the present disclosure; and

FIG. 4A is a flowchart of steps performed during an operational phase of the phacoemulsification procedure, FIG. 4B is a schematic block diagram of elements used during the operational phase, and FIG. 4C illustrates simulated results acquired during the operational phase, according to an example of the present disclosure.

### DESCRIPTION OF EXAMPLES

### Overview

In a phacoemulsification procedure, assumed herein to be performed to remove the lens of a patient's eye, a surgeon uses a phacoemulsification handpiece to insert a hollow needle into the eye. The needle is vibrated ultrasonically, causing the lens to break into particles, and an aspiration pump aspirates the particles from the eye via an aspiration line from the needle. In order to maintain the intraocular pressure (IOP) of the eye within acceptable bounds while the aspiration occurs, an irrigation pump separately irrigates the eye via an irrigation line to the needle. The irrigation flow and the aspiration flow need to be set so as to prevent the acceptable bounds being exceeded, since breaching either an upper or a lower IOP bound may cause irreparable damage to the eye.

The IOP may be monitored by a first pressure sensor, herein termed an irrigation pressure sensor, in the irrigation line. The IOP may also be monitored by a second pressure sensor, herein termed an aspiration pressure sensor, in the aspiration line. Either sensor may be located anywhere along the irrigation line or the aspiration line, in or near a handpiece, or in the console. In an example, the aspiration pressure sensor is part of an anti-vacuum system (AVS), described below, in the handpiece.

During aspiration, an occlusion to the handpiece needle, caused by lens particles being sucked to the needle, may occur. The occlusion limits the aspiration flow, so that the IOP may increase. The AVS limits vacuum transfer into the eye when the occlusion to the handpiece needle breaks. However, when the occlusion breaks and the AVS operates, the IOP may reduce.

Typically, during the procedure, the site being operated on is irrigated continuously, and the aspiration is toggled to activate the aspiration flow, as required by the surgeon, typically by the surgeon activating a foot pedal. Absent an occlusion, the toggling typically reduces the IOP, and this may be detected by the irrigation pressure sensor and/or the aspiration pressure sensor. When IOP reduction is detected, the irrigation flow rate is increased to counteract the reduction.

While the IOP changes described above may be compensated for, typically with a negative feedback loop, by changes in the irrigation and/or the aspiration flow rates provided by their respective pumps, the inventors have found that there may still be unwanted oscillations in the IOP, and these oscillations may be enhanced because of the small volume of the surgical site. Such oscillations, which may in some cases even exceed the IOP bounds described above, may be in response to inherent flow rate changes imparted into the liquids being pumped. (The pumps may impart oscillatory translational and/or rotational motion components into the liquids.) The oscillations may not be adequately compensated for, because, for example, of timing delays caused by the lengths of tubing between the pumps and the handpiece.

To reduce the oscillations, examples of the disclosure measure frequency components of the IOP oscillations, as well as phases of the components. The components and their phases may be measured both before the phacoemulsification procedure, as well as during the procedure. During the procedure a processor applies control signals to the irrigation and/or the aspiration pumps, and the control signals are configured to drive the pumps at the measured frequencies, but in antiphase.

By driving the pumps in antiphase to reduce unwanted oscillations, rather than using a negative feedback loop, examples of the disclosure overcome the problems, referred to above, causing the oscillations.

### SYSTEM DESCRIPTION

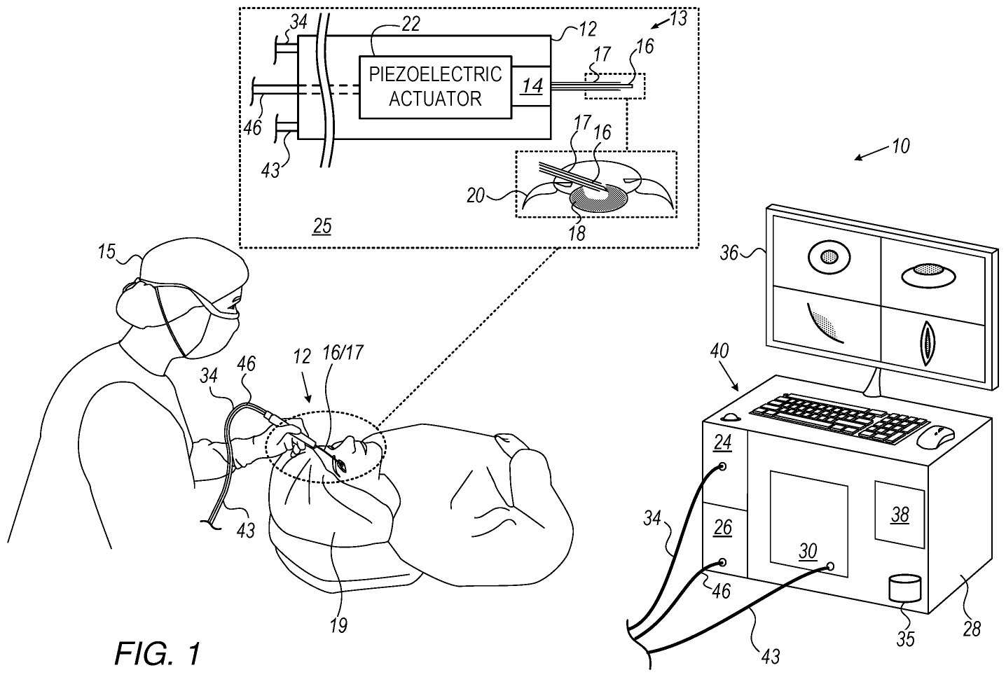

FIG. 1 is a pictorial view of a phacoemulsification apparatus 10, constructed to operate in accordance with an example of the present disclosure, being used for a phacoemulsification procedure.

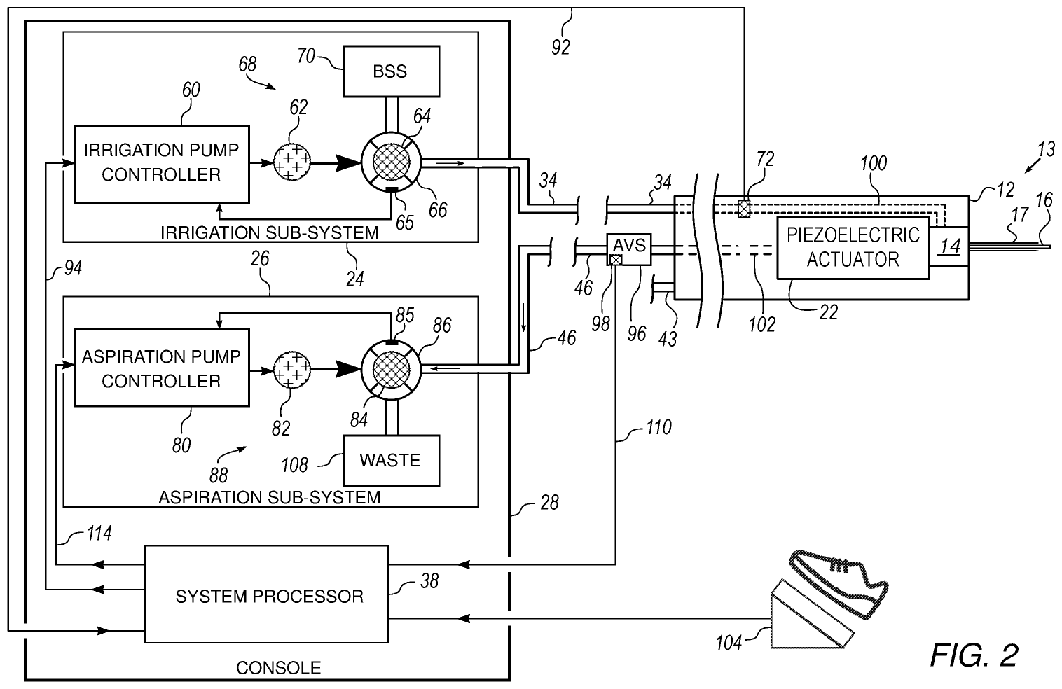

FIG. 1 includes an inset 25, and as shown in the figure and the inset apparatus 10 includes a phacoemulsification probe/handpiece 12 comprising, at a distal end 13 of the probe, a needle 16 and a coaxial irrigation sleeve 17 that at least partially surrounds the needle 16 and that creates a fluid pathway between the external wall of the needle 16 and the internal wall of the irrigation sleeve 17. Needle 16 is configured to be inserted into a lens capsule 18 of an eye 20 of a patient 19. Needle 16 is mounted on a horn 14 of probe 12 and is shown in inset 25 as a straight needle. However, any suitable needle may be used with the phacoemulsification probe 12, for example, a curved or bent tip needle commercially available from Johnson & Johnson Surgical Vision, Inc., Irvine, CA, USA. A physician 15 holds handpiece 12 so as to perform a phacoemulsification procedure on the eye 20 of patient 19. The physician may activate the handpiece using a foot pedal, described below with reference to FIG. 2.

Handpiece 12 comprises a piezoelectric actuator 22, which is configured to vibrate horn 14 and needle 16 in one or more vibration modes of the combined horn and needle. During the phacoemulsification procedure the vibration of needle 16 is used to break a cataract into small pieces.

Elements of apparatus 10 are under overall control of a processor 38 in a console 28. Functions of processor 38 are describe in more detail below.

During the phacoemulsification procedure, an irrigation sub-system 24 in console 28 pumps irrigation fluid to irrigation sleeve 17 so as to irrigate the eye 20. The fluid is pumped via an irrigation tubing line 34 running from the console 28 to the probe 12. An aspiration sub-system 26, also located in console 28, aspirates eye fluid and waste matter (e.g., emulsified parts of the cataract) from the patient's eye via needle 16. Aspiration sub-system 26 comprises a pump which produces a vacuum that is connected from the aspiration sub-system 26 to probe 12 by a vacuum aspiration tubing line 46.

Irrigation sub-system 24 and aspiration sub-system 26 are both under overall control of processor 38. Some or all of the functions of processor 38 may be combined in a single physical component or, alternatively, implemented using multiple physical components. The physical components may comprise hard-wired or programmable devices, or a combination of the two. The functions and structure of irrigation sub-system 24 and aspiration sub-system 26 are described with respect to FIG. 2 below.

FIG. 2 is a schematic block diagram illustrating irrigation sub-system 24 and aspiration sub-system 26, according to an example of the present disclosure. Irrigation sub-system 24 comprises an irrigation pump 68, herein by way of example assumed to comprise a progressive cavity pump (PCP) having an internal rotor 64 and an external stator 66. Pump 68 is driven by a motor 62, which is controlled by an irrigation pump controller 60. Pump 68 has an encoder 65, which is connected to controller 60, enabling the controller to be able to register the position of rotor 64 with respect to stator 66. Using signals from encoder 65, controller 60 can control both the frequency and phase of operation of pump 68.

Irrigation tubing line 34 is connected to an irrigation channel 100 incorporated in handpiece 12. Irrigation fluid, typically a balanced salt solution, is pumped, from an irrigation fluid reservoir 70, by pump 68 through tubing line 34 and irrigation channel 100 to irrigation sleeve 17. A pressure sensor 72, illustrated as being located in irrigation channel 100, is coupled with the irrigation fluid so as to determine a pressure of the irrigation fluid. The sensor provides a measure of the IOP, and a connecting line 92 indicates that the signal generated by the pressure sensor is provided to system processor 38. It will be understood that pressure sensor 72 may be coupled with the irrigation fluid in locations other than irrigation channel 100, such as tubing line 34 or an AVS 96 (described below).

As described below, processor 38 uses the input from sensor 72, as well as other inputs, to provide a driving signal to pump controller 60, as shown by a connecting line 94.

Aspiration sub-system 26 comprises an aspiration pump 88, herein by way of example assumed to comprise a PCP having an internal rotor 84 and an external stator 86. Pump 88 is driven by a motor 82, which is controlled by an irrigation pump controller 80. Pump 88 has an encoder 85, which is connected to controller 80, enabling the controller to be aware of and use the position of rotor 84 with respect to stator 86. Using signals from encoder 85, controller 80 can control both the frequency and phase of operation of pump 88.

Aspiration tubing line 46 is connected to an aspiration channel 102 in handpiece 12. When operative, pump 88 aspirates matter acquired by needle 16, via channel 102 and tubing 46, to a waste matter container 108. There is an anti-vacuum system (AVS) or chamber stability system (CSS) 96 in line 46 that may be coupled with irrigation tubing line 34 and that is configured to limit the fluid and lens particles sucked out of the eye of the patient via the needle 16 when an occlusion breaks (also known as post occlusion surge).

AVS 96 has a pressure sensor 98 incorporated in the AVS and coupled with the aspiration fluid, and, as for pressure sensor 72, the signal generated by sensor 98 is indicative of the IOP. Pressure sensor 98 may be coupled with the aspiration fluid by being incorporated into entities, such as aspiration tubing line 46, other than AVS 96. A connecting line 110 provides the signal from sensor 98 to system processor 38. Processor 38 is configured to use the signal from sensor 98, as well as other inputs as described below, to provide a driving signal to aspiration pump controller 80, as shown by connecting line 114.

During the procedure, physician 15 operates the irrigation and aspiration sub-systems (24, 26), using a sub-system user interface. In an example of the disclosure a foot-pedal 104 acts as the sub-system user interface, and in a disclosed example the foot-pedal has four positions: a first position where the foot-pedal is not activated such that neither sub-system is activated, a second position where the irrigation sub-system alone is activated, a third position where both the irrigation and the aspiration sub-system are activated, and a fourth position where ultrasound is activated in addition to the activated irrigation and aspiration sub-systems.

Returning to FIG. 1, in some examples, at least some of the functions of processor 38 may be carried out by suitable software stored in a memory 35. The software may be downloaded to a device in electronic form, over a network, for example. Alternatively, or additionally, the software may be stored in tangible, non-transitory computer-readable storage media, such as optical, magnetic, or electronic memory.

Console 28 comprises a piezoelectric drive module 30, which is coupled with piezoelectric actuator 22, via processor 38, using electrical wiring running in a cable 43.

Processor 38 may receive user-based commands via a system user interface 40, which may include setting and/or adjusting a vibration mode and/or a frequency of piezoelectric actuator 22, setting and/or adjusting a stroke amplitude of needle 16, and setting and/or adjusting a default irrigation rate and a default aspiration rate of irrigation sub-system 24 and aspiration sub-system 26. Additionally, or alternatively, processor 38 may receive user-based commands from controls located in handpiece 12, to, for example, select a trajectory for needle 16.

Processor 38 may present results of the phacoemulsification procedure on a display 36. In an example, user interface 40 and display 36 may be one and the same, such as a touch screen graphical user interface.

The procedure illustrated in FIG. 1 may include further elements, which are omitted for clarity of presentation. For example, physician 15 typically performs the procedure using a stereo-microscope or magnifying glasses, neither of which are shown. Physician 15 may use other surgical tools, in addition to probe 12, which are also not shown to maintain clarity and simplicity.

Because, during the phacoemulsification procedure referred to herein, there is continuous irrigation as well as intermittent aspiration of eye 20, the IOP in the eye varies. Examples of the present disclosure mitigate these variations, as described below.

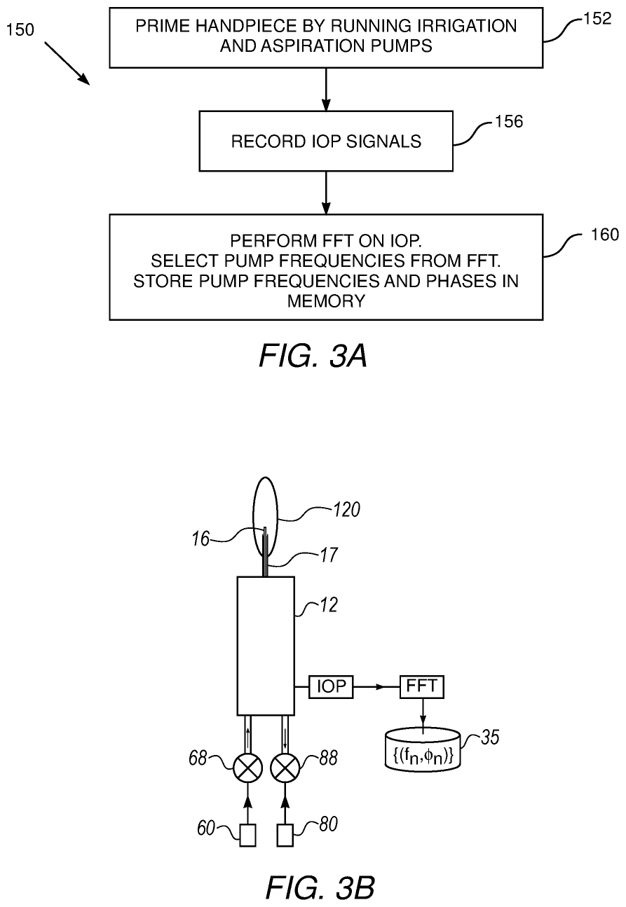

FIG. 3A is a flowchart 150 of steps performed during a handpiece priming phase of the phacoemulsification procedure, and FIG. 3B is a schematic block diagram of elements of apparatus 10 used during the priming phase, according to an example of the present disclosure.

Before an operational phase of the procedure, when the needle of the handpiece is inserted into the patient's eye, handpiece 12 is primed, by enclosing needle 16 and sleeve 17 in a test chamber 120. Test chamber 120 ensures that the output of the irrigation pump forms the input of the aspiration pump, i.e., that the irrigation fluid pumped from the irrigation pump corresponds to the aspiration fluid pumped by the aspiration pump.

In a first step 152 of the priming phase processor 38 activates both irrigation pump 68 and aspiration pump 88, via controllers 60 and 80, to pump liquid through needle 16 and sleeve 17. The priming removes any air bubbles that may be present in the irrigation and aspiration lines. In an example, during the priming processor 38 sequentially operates, at different times, both the pumps at a common rate r₁, r₂, . . . rₙ, r₁≠r₂≠rₙ, where n is an index of the pump rate. I.e., at any given time, the irrigation pump is operated at a rate rₘ, and the aspiration pump is also operated at rate rₘ.

In a recordation step 156, while the priming of step 152 is being performed, processor 38 records and stores the signals from pressure sensor 72 in the irrigation channel, and from pressure sensor 98 in the aspiration tubing line.

In an analysis step 160 which concludes the flowchart, and which may be performed during or after the priming of the handpiece, processor 38 converts the signals from sensors 72 and 98 to IOP values. The processor then performs a fast Fourier transform (FFT) on the pressure values, to identify frequency components of the pressures, as well as amplitudes and phases of the identified components. The FFT is performed for each of the different pump rates performed in step 152. Typically, each frequency component has a value between approximately 0.1 Hz and approximately 10 Hz, but the components may be outside this range.

For a given pump rate, the components determined by the FFT typically include noisy components. To differentiate from the noise, in an example, for each given pump rate processor 38 finds the average amplitude of the frequency components and identifies components above a preset threshold amplitude. In an example, the preset threshold amplitude is ten times the average amplitude, but other examples may have the preset threshold larger or small than ten times the average.

In a disclosed example, described hereinbelow, the number of components identified in analysis step 160 is assumed to be two, and processor 38 is configured to identify, by inspecting amplitudes of the components, the two largest components greater than the threshold.

Those having ordinary skill in the art will be able to adapt the description, mutatis mutandis, for numbers of components different from two, i.e., when only one component is identified, or when three or more components are identified.

For each given pump rate processor 38 stores in memory 35 the pair of frequency values of each of the identified components, herein termed pump frequencies, as well as the phase of the components, as pairs of ordered frequency-phase values:

$$\{ [ ( {f}_{11} , {φ}_{11} ) , ( {f}_{21} , {φ}_{21} ) ] , [ ( {f}_{12} , {φ}_{12} ) , ( {f}_{22} , {φ}_{22} ) ] … [ ( {f}_{1 n} , {φ}_{1 n} ) , ( {f}_{2 n} , {φ}_{2 n} ) ] \}$$

where n is the index indicative of the pump rate at which the frequency and phase pairs were acquired.

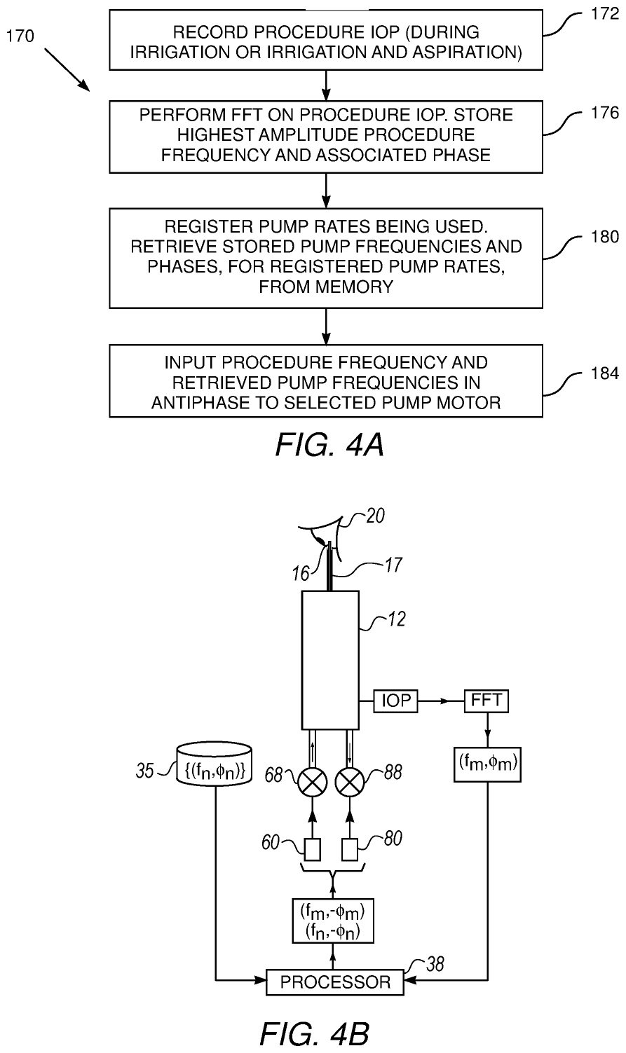

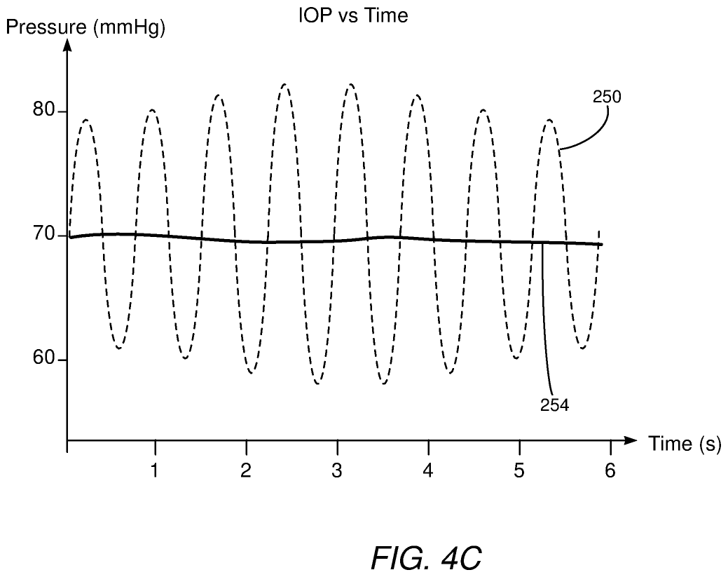

Processor 38 uses the stored frequency and phase values in an operational phase of the procedure, described below with reference to FIGS. 4A, 4B, and 4C.

FIG. 4A is a flowchart 170 of steps performed during an operational phase of the phacoemulsification procedure, FIG. 4B is a schematic block diagram of elements of apparatus 10 used during the operational phase, and FIG. 4C illustrates simulated results acquired during the operational phase, according to an example of the present disclosure.

In an initial step 172, physician 15 inserts needle 16 and sleeve 17 into eye 20 of the patient and activates the irrigation and aspiration pumps (68, 88). Typically, the irrigation pump 68 is activated substantially continuously, and the aspiration pump 88 is activated as required. Physician 15 may use foot-pedal 104 for the activations. It will be understood that, in contrast to the priming phase, in the operational phase the output of the irrigation pump 68 does not form the input of the aspiration pump 88. Rather, the output of the irrigation pump 68 is a liquid directed into eye 20, and the input of the aspiration pump 88 comprises particles and/or fluid of eye 20.

While the pumps are activated in the procedure, processor 38 acquires the signals from pressure sensor 72 in the irrigation channel 100, and from pressure sensor 98 in the aspiration tubing line 46. The processor 38 converts the acquired signals to IOP values, which are herein termed procedure IOP values, and stores the procedure IOP values.

In a processing step 176 implemented during the procedure, processor 38 performs an FFT on the stored procedure IOP values. From the FFT, the processor 38 identifies a frequency component with the highest amplitude, herein termed the procedure frequency. The processor 38 stores the procedure frequency, and the phase associated with the procedure frequency, as an ordered pair (fₘ, ϕₘ).

In a registration step 180, processor 38 registers the pump rates being used, i.e., the rate at which controller 60 is driving irrigation pump 68, and, if aspiration is being implemented, the rate at which controller 80 is driving the aspiration pump 88. The rates may be identified by indexes 1, 2, . . . , n described above. The processor then retrieves from memory 35 the pump frequencies and phases, associated with the registered pump rates, that have been stored in step 160 of flowchart 150. Thus, for a pump rate rₙ, the processor retrieves (f₁ₙ, ϕ₁ₙ) and (f₂ₙ, ϕ₂ₙ).

In a pump input step 184, processor 38 provides control signals to controller 60 or controller 80 to drive their respective pumps, irrigation pump 68 and aspiration pump 88, at the frequencies retrieved in step 180, and at the procedure frequency stored in step 176. However, the signals are configured to drive the pumps in antiphase to the retrieved and stored phases. I.e., the control signals provide the frequencies and phases: (fₘ,−ϕₘ), (f₁ₙ,−ϕ₁ₙ), and (f₂ₙ,−ϕ₂ₙ) If only irrigation is being used, then the frequencies and antiphase values are provided to irrigation controller 60; if irrigation and aspiration are being used then the frequencies and antiphase values may be provided to either controller, and in one example the controller is selected to be aspiration controller 80.

FIG. 4C illustrates simulated IOP vs. time results. A graph 250 has a mean pressure value of approximately 70 mmHg, and peak-peak oscillations of approximately 20 mmHg. The oscillations have a frequency of approximately 1.3 Hz. Graph illustrates the expected IOP variation if the steps of flowchart 170 are not implemented during a phacoemulsification procedure.

A graph 254 illustrates IOP vs. time if flowchart 170 is implemented.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g., “about 90%” may refer to the range of values from 81% to 99%.

### Examples

Example 1. A phacoemulsification system, comprising: a phacoemulsification probe (12) having an irrigation channel (100) and an aspiration channel (102), and a distal end (13) comprising a needle (16) and a sleeve (17) configured to be inserted into an eye; an irrigation pump (68) configured to pump irrigation fluid via the irrigation channel and the distal end into the eye; an aspiration pump (88) configured to pump aspiration fluid via the distal end and the aspiration channel from the eye; a first pressure sensor (72) coupled with the irrigation fluid and configured to provide a first signal indicative of intraocular pressure (IOP) in the eye; a second pressure sensor (98) coupled with the aspiration fluid and configured to provide a second signal indicative of the IOP in the eye; and a system processor (38) configured to: receive the first signal and the second signal, and responsively to at least one of the first signal and the second signal, identify at least one frequency and an associated at least one phase of the IOP, and pump at least one of the aspiration pump and the irrigation pump at the identified at least one frequency and in antiphase to the identified at least one phase.

Example 2. The system according to example 1, wherein the system processor is further configured to convert the received first and second signals to IOP values and perform a fast Fourier transform (FFT) on the IOP values, and wherein identifying the at least one frequency and the at least one phase of the IOP comprises selecting from the FFT an IOP value having a maximum amplitude.

Example 3. The system according to any of examples 1 to 2, further comprising a test chamber (120) configured to receive the needle and the sleeve, the system processor being configured to identify the at least one frequency and the at least one phase in a priming phase of a phacoemulsification procedure, wherein in the priming phase the needle and the sleeve are placed in the test chamber so that the irrigation fluid pumped by the irrigation pump comprises the aspiration fluid pumped by the aspiration pump.

Example 4. The system according to example 3, wherein in the priming phase the irrigation pump and the aspiration pump pump at a common first rate in a first time period, and in a common second rate, different from the common first rate, in a second time period different from the first time period, and wherein the system processor is configured to identify a first frequency and an associated first phase in the first time period, and a second frequency and an associated second phase in the second time period.

Example 5. The system according to any of examples 1 to 4, wherein the system processor is further configured to identify the at least one frequency and the at least one phase in an operational phase of a phacoemulsification procedure, wherein in the operational phase the needle and the sleeve are inserted into the eye so that the irrigation fluid is pumped into the eye, and the aspiration fluid comprises entities of the eye pumped therefrom.

Example 6. The system according to example 5, wherein identifying the at least one frequency and the associated at least one phase comprises storing the at least one frequency and the associated at least one phase in a priming phase of the phacoemulsification procedure performed prior to the operational phase, and retrieving the stored at least one frequency and the associated at least one phase during the operational phase.

Example 7. The system according to example 6, wherein in the priming phase the irrigation pump and the aspiration pump pump at a common rate, and wherein in the operational phase the system processor is configured to detect that at least one of the irrigation pump and the aspiration pump is pumping at the common rate, and in response to the detection retrieve the stored at least one frequency and the associated at least one phase.

Example 8. The system according to any of examples 1 to 7, wherein the irrigation pump comprises an encoder (65) configured to provide an encoding signal to the system processor, and wherein in response to receiving the encoding signal, the system processor is configured to pump the irrigation pump at the identified at least one frequency and in antiphase to the identified at least one phase.

Example 9. The system according to any of examples 1 to 8, wherein the aspiration pump comprises an encoder (85) configured to provide an encoding signal to the system processor, and wherein in response to receiving the encoding signal, the system processor is configured to pump the aspiration pump at the identified at least one frequency and in antiphase to the identified at least one phase.

Example 10. The system according to an of examples 1 to 9, wherein the irrigation pump and the aspiration pump comprise progressive cavity pumps.

Example 11. A method, comprising: providing a phacoemulsification probe (12) having an irrigation channel (100) and an aspiration channel (102), and a distal end (13) comprising a needle (16) and a sleeve (17) configured to be inserted into an eye; configuring an irrigation pump (68) to pump irrigation fluid via the irrigation channel and the distal end into the eye; configuring an aspiration pump (88) to pump aspiration fluid via the distal end and the aspiration channel from the eye; coupling a first pressure sensor (72) with the irrigation fluid so as to provide a first signal indicative of intraocular pressure (IOP) in the eye; coupling a second pressure sensor (98) with the aspiration fluid so as to provide a second signal indicative of the IOP in the eye; receiving the first signal and the second signal, and responsively to at least one of the first signal and the second signal, identifying at least one frequency and an associated at least one phase of the IOP; and pumping at least one of the aspiration pump and the irrigation pump at the identified at least one frequency and in antiphase to the identified at least one phase.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A phacoemulsification system, comprising:
a phacoemulsification probe having an irrigation channel and an aspiration channel, and a distal end comprising a needle and a sleeve configured to be inserted into an eye;
an irrigation pump configured to pump irrigation fluid via the irrigation channel and the distal end into the eye;
an aspiration pump configured to pump aspiration fluid via the distal end and the aspiration channel from the eye;
a first pressure sensor coupled with the irrigation fluid and configured to provide a first signal indicative of intraocular pressure (IOP) in the eye;
a second pressure sensor coupled with the aspiration fluid and configured to provide a second signal indicative of the IOP in the eye; and
a system processor configured to:
receive the first signal and the second signal, and
responsively to at least one of the first signal and the second signal,
identify at least one frequency and an associated at least one phase of the IOP, and
pump at least one of the aspiration pump and the irrigation pump at the identified at least one frequency and in antiphase to the identified at least one phase.

2. The system according to claim 1, wherein the system processor is further configured to convert the received first and second signals to IOP values and perform a fast Fourier transform (FFT) on the IOP values, and wherein identifying the at least one frequency and the at least one phase of the IOP comprises selecting from the FFT an IOP value having a maximum amplitude.

3. The system according to claim 1, further comprising a test chamber configured to receive the needle and the sleeve, the system processor being configured to identify the at least one frequency and the at least one phase in a priming phase of a phacoemulsification procedure, wherein in the priming phase the needle and the sleeve are placed in the test chamber so that the irrigation fluid pumped by the irrigation pump comprises the aspiration fluid pumped by the aspiration pump.

4. The system according to claim 3, wherein in the priming phase the irrigation pump and the aspiration pump pump at a common first rate in a first time period, and in a common second rate, different from the common first rate, in a second time period different from the first time period, and wherein the system processor is configured to identify a first frequency and an associated first phase in the first time period, and a second frequency and an associated second phase in the second time period.

5. The system according to claim 1, wherein the system processor is further configured to identify the at least one frequency and the at least one phase in an operational phase of a phacoemulsification procedure, wherein in the operational phase the needle and the sleeve are inserted into the eye so that the irrigation fluid is pumped into the eye, and the aspiration fluid comprises entities of the eye pumped therefrom.

6. The system according to claim 5, wherein identifying the at least one frequency and the associated at least one phase comprises storing the at least one frequency and the associated at least one phase in a priming phase of the phacoemulsification procedure performed prior to the operational phase, and retrieving the stored at least one frequency and the associated at least one phase during the operational phase.

7. The system according to claim 6, wherein in the priming phase the irrigation pump and the aspiration pump pump at a common rate, and wherein in the operational phase the system processor is configured to detect that at least one of the irrigation pump and the aspiration pump is pumping at the common rate, and in response to the detection retrieve the stored at least one frequency and the associated at least one phase.

8. The system according to claim 1, wherein the irrigation pump comprises an encoder configured to provide an encoding signal to the system processor, and wherein in response to receiving the encoding signal, the system processor is configured to pump the irrigation pump at the identified at least one frequency and in antiphase to the identified at least one phase.

9. The system according to claim 1, wherein the aspiration pump comprises an encoder configured to provide an encoding signal to the system processor, and wherein in response to receiving the encoding signal, the system processor is configured to pump the aspiration pump at the identified at least one frequency and in antiphase to the identified at least one phase.

10. A method, comprising:
providing a phacoemulsification probe having an irrigation channel and an aspiration channel, and a distal end comprising a needle and a sleeve configured to be inserted into an eye;
configuring an irrigation pump to pump irrigation fluid via the irrigation channel and the distal end into the eye;
configuring an aspiration pump to pump aspiration fluid via the distal end and the aspiration channel from the eye;
coupling a first pressure sensor with the irrigation fluid so as to provide a first signal indicative of intraocular pressure (IOP) in the eye;
coupling a second pressure sensor with the aspiration fluid so as to provide a second signal indicative of the IOP in the eye;
receiving the first signal and the second signal, and responsively to at least one of the first signal and the second signal, identifying at least one frequency and an associated at least one phase of the IOP; and
pumping at least one of the aspiration pump and the irrigation pump at the identified at least one frequency and in antiphase to the identified at least one phase.

11. The method according to claim 10, further comprising converting the received first and second signals to IOP values, performing a fast Fourier transform (FFT) on the IOP values, and wherein identifying the at least one frequency and the at least one phase of the IOP comprises selecting from the FFT an IOP value having a maximum amplitude.

12. The method according to claim 10, further comprising configuring a test chamber to receive the needle and the sleeve, and identifying the at least one frequency and the at least one phase in a priming phase of a phacoemulsification procedure performed on the eye, wherein in the priming phase the needle and the sleeve are placed in the test chamber so that the irrigation fluid pumped by the irrigation pump comprises the aspiration fluid pumped by the aspiration pump.

13. The method according to claim 12, wherein in the priming phase the irrigation pump and the aspiration pump pump at a common first rate in a first time period, and in a common second rate, different from the common first rate, in a second time period different from the first time period, the method further comprising identifying a first frequency and an associated first phase in the first time period, and a second frequency and an associated second phase in the second time period.

14. The method according to claim 10, further comprising identifying the at least one frequency and the at least one phase in an operational phase of a phacoemulsification procedure, wherein in the operational phase the needle and the sleeve are inserted into the eye so that the irrigation fluid is pumped into the eye, and the aspiration fluid comprises entities of the eye pumped therefrom.

15. The method according to claim 14, wherein identifying the at least one frequency and the associated at least one phase comprises storing the at least one frequency and the associated at least one phase in a priming phase of the phacoemulsification procedure performed prior to the operational phase, and retrieving the stored at least one frequency and the associated at least one phase during the operational phase.

16. The method according to claim 15, wherein in the priming phase the irrigation pump and the aspiration pump pump at a common rate, the method further comprising, in the operational phase, detecting that at least one of the irrigation pump and the aspiration pump pump at the common rate, and in response to the detection retrieving the stored at least one frequency and the associated at least one phase.

17. The method according to claim 10, wherein the irrigation pump comprises an encoder configured to provide an encoding signal, the method further comprising, in response to receiving the provided encoding signal, pumping the irrigation pump at the identified at least one frequency and in antiphase to the identified at least one phase.

18. The method according to claim 10, wherein the aspiration pump comprises an encoder configured to provide an encoding signal, the method further comprising, in response to receiving the provided encoding signal, pumping the aspiration pump at the identified at least one frequency and in antiphase to the identified at least one phase.
